(19) United States
(12) Patent Application Publication
(10) Pub. No.: US 2024/0174947 A1
(43) Pub. Date: May 30, 2024

(54) SURFACTANT COMPOSITIONS

(71) Applicant: LANKEM LTD., Dukinfield Cheshire (GB)

(72) Inventors: Sean Graham HODGKINSON, Dukinfield Cheshire (GB); John ADAMSON, Dukinfield Cheshire (GB)

(21) Appl. No.: 18/277,904

(22) PCT Filed: Feb. 17, 2022
(86) PCT No.: PCT/GB2022/050425

§ 371 Date: Aug. 18, 2023

(30) Foreign Application Priority Data
Feb. 19, 2021 (GB) 2102402.1

(51) Int. Cl. C11D 1/28; A61K 8/46; A61Q 19/10

(52) U.S. Cl. CPC C11D 1/28; A61K 8/466; A61Q 19/10

(57) ABSTRACT

A mild surfactant composition for use in personal care and/or detergent compositions. The composition comprises a surfactant which is mild and/or non-irritating to skin. The mild surfactant is the reaction product of (a) an epoxidised carboxylic acid ester; and (b) a compound including at least one reactive alcohol and/or amino functional group. The components (a) and (b) are suitably obtained from renewable sources. A use of such surfactants in mild surfactant compositions is also provided.

### FIELD

The present invention relates to a mild surfactant composition and the use of specific surfactant compounds in mild surfactant compositions. In particular, the invention relates to surfactant compounds prepared from renewable sources which are mild to skin.

### BACKGROUND

In recent times there has been mounting public and regulatory pressure for industrial processes to reduce their environmental impact, including processes using surfactants. One way of reducing the environmental impact of an industrial process is to reduce its dependency on non-renewable materials. Therefore, it is desirable that surfactants can be obtained from renewable sources.

Due to the wide variety of possible uses of surfactant compounds, and the huge variability of the compositions in which they are included, there is a continuing need to provide surfactant compounds having differing structures which can be varied according to the intended use of the surfactant. There is also a continuing need for new surfactant compounds which may exhibit different properties to existing compounds.

Surfactants are used in a wide range of both domestic and industrial applications where low irritancy to skin and eyes is essential or at the very least beneficial. This can include applications where the surface-active agent containing products intentionally comes into contact with the skin, such as personal care or cosmetic products, or applications where the prevention of contact cannot be guaranteed, such as in household or industrial cleaning products, for example laundry detergents or dish washing compositions.

It would be beneficial if a surfactant could be provided which is mild or non-irritating and which can be obtained from renewable sources so that the environmental impact of such products can be improved.

### SUMMARY OF THE INVENTION

It is one aim of the present invention, amongst others, to provide a mild surfactant composition and a use of a surfactant as a mild surfactant that addresses at least one disadvantage of the prior art, whether identified here or elsewhere, or to provide an alternative to existing mild surfactant compositions. For instance, it may be an aim of the present invention to provide a personal care or laundry detergent composition which is mild to the skin and eyes using a mild surfactant which may also be prepared from renewable sources.

According to aspects of the present invention, there is provided a mild surfactant composition and use as set forth in the appended claims. Other features of the invention will be apparent from the dependent claims, and from the description which follows.

According to a first aspect of the present invention, there is provided a mild surfactant composition comprising a surfactant which is the reaction product of:
(a) an epoxidised carboxylic acid ester; and
(b) a compound including at least one reactive alcohol and/or amino functional group.

According to a second aspect of the present invention, there is provided a use of a reaction product of:
(a) an epoxidised carboxylic acid ester; and
(b) a compound including at least one reactive alcohol and/or amino functional group,
   as a surfactant in a mild surfactant composition.

The surfactant and surfactant composition according to the first and second aspects is referred to as mild. Mild surfactants and mild surfactant compositions are known in the relevant arts and these terms refer to surfactants and surfactant compositions which have low irritancy to the skin and/or eyes, suitably of a human. The mildness and/or low irritancy can be measured by several known methods such as the Reconstructed Human Epidermis ET50 method, the EpiOculuar (RTM) test or the Zein test. Suitably the surfactant and surfactant composition of the first and second aspects are classified as mild, very mild or non-irritating in the above-mentioned tests, or in an equivalent or similar proprietary test. Suitably the surfactant and surfactant composition are classified as non-irritating in such tests, suitably in a Reconstructed Human Epidermis ET50 method. Therefore the surfactant and surfactant composition may be referred to as a non-irritating surfactant and non-irritating surfactant composition.

The use of the second aspect of the reaction product of (a) and (b) as a surfactant in a mild surfactant composition may be alternatively referred to as the use of this reaction product as a mild (or non-irritating) surfactant in a surfactant composition, suitably a personal care composition or a detergent composition. Suitably the reaction product provides the surfactant properties required of such surfactant compositions whilst not contributing to or causing skin or eye irritation to a user, which can be caused by some widely used surfactants. Suitably the reaction product of (a) and (b) is the main surfactant component in such surfactant compositions. Suitably no non-mild surfactants are present in the surfactant compositions.

Suitably the use of the second aspect is achieved by incorporating the reaction product of (a) and (b) into a surfactant composition to provide a mild surfactant composition, wherein the reaction product provides the surfactant properties required of the composition whilst not introducing irritancy which may be caused by known surfactants. Suitable amounts of the reaction product to use, other further ingredients to include in the mild surfactant composition and the types of composition which would benefit from the use of this reaction product would be known to the skilled person, in light of the present disclosure.

Therefore the present invention may also provide a method of forming a mild surfactant composition, the method comprising the steps of:
(i) preparing a mild surfactant by reacting:
   (a) an epoxidised carboxylic acid ester; and
   (b) a compound including at least one reactive alcohol and/or amino functional group;
(ii) incorporating the mild surfactant into a composition to provide the mild surfactant composition.

The inventors have surprisingly found that the reaction products of (a) and (b) are not only effective surfactants obtainable from renewable sources but are also mild and/or non-irritating in relevant tests. These reaction products are therefore particularly suitable for use in surfactant compositions which may come into contact with the skin and possibly the eyes of users, such as personal care compositions and detergent compositions, for example laundry detergents. The present invention can therefore provide mild/non-irritating surfactants which may have an improved environmental profile compared to known mild surfactants. These surfactants may therefore improve the mildness and environmental profile of such surfactant compositions, when used as the main surfactant component these surfactant compositions.

### Component (a)

Component (a) comprises an epoxidised carboxylic acid ester. The epoxidised carboxylic acid ester (a) may comprise an ester of a monocarboxylic acid or a polycarboxylic acid, for example a dicarboxylic acid. In embodiments in which component (a) comprises an ester of a polycarboxylic acid, suitably each acid group is esterified.

Preferably component (a) comprises an ester of formula RCOOR¹ in which R is a hydrocarbyl group including an epoxy functional group and R¹ is a hydrocarbyl group.

R is suitably an alkyl, alkenyl or aralkyl group comprising at least one epoxy functional group and R¹ is suitably an alkyl or aryl group.

R comprises at least one epoxy functional group. It may comprise more than one epoxy functional group. R is suitably an alkyl, alkenyl or aralkyl group having one or more epoxy substituents, preferably one or two epoxy substituents. Preferably R is an epoxy substituted alkyl or alkenyl group.

In preferred embodiments, the or each epoxy functional group is a secondary epoxy group. By secondary epoxy group, we mean an epoxy group wherein both carbon atoms of the epoxide ring are bound to a further carbon atom, i.e. the epoxy moiety is preferably not at a terminal position.

Suitably the epoxidised carboxylic acid ester (a) comprises one to four, preferably one to three, more preferably one or two epoxy groups. In some preferred embodiments, the epoxidised carboxylic acid ester (a) contains one epoxy group. In some preferred embodiments, the epoxidised carboxylic acid ester (a) contains two epoxy groups.

In some embodiments R may include one or more further substituents in addition to the one or more epoxy substituents. In preferred embodiments the one or more epoxy groups are the only substituents of the alkyl, alkenyl or aralkyl group R.

In some embodiments R is an epoxy substituted alkenyl group. Suitably the alkenyl group may comprise one or more double bonds, for example one, two or three double bonds, suitably one or two double bonds.

Preferably R is an epoxy substituted alkyl group.

Preferably R is an aliphatic group, more preferably an unbranched aliphatic group.

Suitably R has at least 6 carbon atoms, preferably at least 8 carbon atoms, more preferably at least 10 carbon atoms. R may have up to 36 carbon atoms, suitably up to 30 carbon atoms, preferably up to 26 carbon atoms, for example up to 24 carbon atoms. In preferred embodiments R has from 6 to 26 carbon atoms, more preferably from 8 to 24 carbon atoms, most preferably from 10 to 22 carbon atoms.

Component (a) may comprise a mixture of compounds including mixtures of homologues in which the groups R have different numbers of carbon atoms. Natural sources of carboxylic acids typically contain mixtures of compounds.

R may be straight chain or branched. Preferably R is straight chain.

Preferably R¹ is an alkyl or aryl group. More preferably R¹ is an alkyl group. Preferably R¹ is an unsubstituted alkyl group.

R¹ has at least 1 carbon atom. Suitably R¹ has up to 22 carbon atoms, preferably up to 20 carbon atoms, more preferably up to 18 carbon atoms. In preferred embodiments R¹ has from 1 to 18 carbon atoms, preferably from 1 to 12 carbon atoms, for example from 1 to 8 carbon atoms. R¹ may be straight chain or branched.

R¹ may be selected from methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 1,1-dimethylpropyl, 1,2-dimethylpropyl, 2,2-dimethylpropyl, 1-ethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, 1-methylhexyl, 2-methylhexyl, 3-methylhexyl, 4-methylhexyl, 5-methylhexyl, 1,1-dimethylpentyl, 1,2-dimethylpentyl, 1,3-dimethylpentyl, 1,4-dimethylpentyl, 2,2-dimethylpentyl, 2,3-dimethylpentyl, 2,4-dimethylpentyl, 3,3-dimethylpentyl, 3,4-dimethylpentyl, 4,4-dimethylpentyl, 1-ethylpentyl, 2-ethylpentyl, 3-ethylpentyl, 1,1,2-trimethylbutyl, 1,1,3-trimethylbutyl, 1,2,2-trimethylbutyl, 1,2,3-trimethylbutyl, 1,3,3-trimethylbutyl, 2,2,3-trimethylbutyl, 2,3,3-trimethylbutyl, 1-ethyl-1-methylbutyl, 1-ethyl-2-methylbutyl, 1-ethyl-3-methylbutyl, 2-ethyl-1-methylbutyl, 2-ethyl-2-methylbutyl, 2-ethyl-3-methylbutyl, 1-propylbutyl, octyl, 1-methylheptyl, 2-methylheptyl, 3-methylheptyl, 4-methylheptyl, 5-methylheptyl, 6-methylheptyl, 1,1-dimethylhexyl, 1,2-dimethylhexyl, 1,3-dimethylhexyl, 1,4-dimethylhexyl, 1,5-dimethylhexyl, 2,2,-dimethylhexyl, 2,3-dimethylhexyl, 2,4-dimethylhexyl, 2,5-dimethylhexyl, 3,3-dimethylhexyl, 3,4-dimethylhexyl, 3,5-dimethylhexyl, 4,4-dimethylhexyl, 4,5-dimethylhexyl, 5,5-dimethylhexyl, 1-ethylhexyl, 2-ethylhexyl, 3-ethylhexyl, 4-ethylhexyl, 1,1,2-trimethylpentyl, 1,1,3-trimethylpentyl, 1,1,4-trimethylpentyl, 1,2,2-trimethylpentyl, 1,2,3-trimethylpentyl, 1,2,4-trimethylpentyl, 1,3,3-trimethylpentyl, 1,3,4-trimethylpentyl, 1,4,4-trimethylpentyl, 2,2,3-trimethylpentyl, 2,2,4-trimethylpentyl, 2,3,3-trimethylpentyl, 2,3,4-trimethylpentyl, 2,4,4-trimethylpentyl, 3,3,4-trimethylpentyl, 3,4,4-trimethylpentyl, 1-ethyl-1-methylpentyl, 1-ethyl-2-methylpentyl, 1-ethyl-3-methylpentyl, 1-ethyl-4-methylpentyl, 2-ethyl-1-methylpentyl, 2-ethyl-2-methylpentyl, 2-ethyl-3-methylpentyl, 2-ethyl-4-methylpentyl, 3-ethyl-1-methylpentyl, 3-ethyl-2-methylpentyl, 3-ethyl-3-methylpentyl, 3-ethyl-4-methylpentyl, 1,1,2,2-tetramethylbutyl, 1,1,2,3-tetramethylbutyl, 1,1,3,3-tetramethylbutyl, 1,2,2,3-tetramethylbutyl, 1,2,3,3-tetramethylbutyl, 1-ethyl-1,2-dimethylbutyl, 1-ethyl-1,3-dimethylbutyl, 1-ethyl-2,2-dimethylbutyl, 1-ethyl-2,3-dimethylbutyl, 1-ethyl-3,3-dimethylbutyl, 2-ethyl-1,1-dimethylbutyl, 2-ethyl-1,2-dimethylbutyl, 2-ethyl-1,3-dimethylbutyl, 2-ethyl-2,3-dimethylbutyl, 2-ethyl-3,3-dimethylbutyl, 1,1-diethylbutyl, 1,2-diethylbutyl, or 2,2-diethylbutyl.

Preferably R¹ is methyl or 2-ethylhexyl. More preferably R¹ is methyl.

Preferably substantially all of the acid groups of the epoxidised carboxylic acid ester are esterified. Preferably the epoxidised carboxylic acid ester suitably contains less than 10%, suitably less than 5%, suitably less 4%, suitably less than 3%, suitably less than 2%, preferably less than 1% free acid groups based on the total number of free acid groups and esterified acid groups.

Suitably component (a) comprises an epoxidised fatty acid ester. Suitably in the fatty acid R is an unbranched aliphatic group, preferably having 4 to 28 carbon atoms, for example 10 to 24 carbon atoms. Suitably component (a) is obtained by the epoxidation of a fatty acid, preferably a naturally occurring fatty acid.

Suitably component (a) is derived from an unsaturated (including polyunsaturated) fatty acid.

Suitably the epoxidised carboxylic acid ester (a) is derived from an unsaturated fatty acid selected from myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, a-linoleic acid, pinolenic acid, eleostearic acid, mead acid, dihomo-γ-linolenic acid, eicosatrienoic acid, stearidonic acid, arachidonic acid, eicosatetraenoic acid, adrenic acid or mixtures thereof.

In preferred embodiments the epoxidised carboxylic acid ester (a) is derived from a naturally occurring unsaturated fatty acid or a combination of naturally occurring unsaturated fatty acids. In one especially preferred embodiment the epoxidised carboxylic acid ester (a) is derived from soybean oil fatty acid.

Therefore component (a) preferably comprises an epoxidised soybean oil ester.

Suitable epoxidised fatty acid esters include epoxidised soybean oil 2-ethylhexyl ester and epoxidised soybean oil methyl ester.

Component (a) may comprise a mixture of compounds.

Naturally occurring fatty acids typically comprise mixtures of compounds.

In embodiments in which component (a) is derived from a polyunsaturated fatty acid, the compound may comprise one or more epoxy groups. In some such embodiments component (a) may comprise compounds in which all of the alkene functional groups are epoxidised and/or it may comprise compounds in which one or more alkene groups are epoxidised and one or more alkene groups remain unreacted.

In some embodiments component (a) may comprise at least one epoxidised carboxylic acid ester containing one epoxy group and at least one epoxidised carboxylic acid ester containing at least two epoxy groups, suitably two epoxy groups.

Some epoxidised carboxylic acid esters (a) are commercially available. Such compounds may also be prepared from unsaturated carboxylic acids and/or esters thereof by methods known to those skilled in the art.

Unsaturated carboxylic acid esters are typically obtained from natural sources by transesterification of naturally occurring triglycerides and/or by esterification of the alcohol and the fatty acid.

Component (a) may be obtained at least partially from renewable sources. Preferably component (a) is obtained from entirely renewable sources.

### Component (b)

Component (b) comprises a compound including at least one reactive alcohol and/or amino functional group. Such groups are suitably able to react with epoxy groups. Suitably the reactive alcohol and/or amino functional groups are especially able to react with secondary epoxy groups.

Preferably component (b) comprises a compound which includes at least two reactive alcohol and/or amino functional groups. In some embodiments the compound may include at least three reactive alcohol and/or amino functional groups. In some embodiments the compound may include at least four reactive alcohol and/or amino functional groups. Component (b) preferably comprises a compound including two reactive alcohol and/or amino functional groups.

In some embodiments component (b) includes a compound having one reactive alcohol functional group. In some embodiments component (b) includes a compound having two reactive alcohol functional groups. In some embodiments component (b) includes a compound having one reactive amino functional group. In some embodiments component (b) includes a compound having two reactive amino functional groups. In some embodiments component (b) includes a compound having one reactive alcohol functional group and one reactive amino functional group.

In embodiments where component (b) includes a compound having reactive alcohol functional groups, the reactive alcohol functional groups are preferably primary alcohol groups.

In embodiments where component (b) includes a compound having reactive amino functional groups, the reactive amino functional groups are suitably primary amino groups or secondary amino groups. In preferred embodiments, the reactive amino functional groups are primary amino groups.

Component (b) preferably comprises a compound of formula (I):

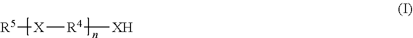

wherein n is 0 or a positive integer; each X is independently O or NH; each group R⁴ is independently an optionally substituted alkylene, alkenylene or arylene group; and R⁵ is hydrogen or an optionally substituted alkyl, alkenyl, aryl, alkaryl or aralkyl group provided that n is not 0 when R⁵ is hydrogen.

In some embodiments n is no more than 300, suitably no more than 250, preferably no more than 200, preferably no more than 150, for example no more than 100.

In some embodiments n is 1.

In some embodiments n is 1 to 20.

In some embodiments n is 10 to 200, for example 10 to 100 or 10 to 50.

Each X may be the same or different.

In some embodiments, each X is O. In some embodiments, each X is NH. In some embodiments, at least one X is O and at least one X is NH.

Each R⁴ is an optionally substituted alkylene, alkenylene or arylene group. Preferably each R 4 is an unsubstituted alkylene, alkenylene or arylene group, more preferably an unsubstituted alkylene or alkenylene group.

When R⁴ is substituted, preferred substituents are amino and hydroxy substituents.

In some embodiments each R⁴ is an unsubstituted alkenylene group. Suitably the alkenylene group may comprise one or more double bonds, for example one or two double bonds, suitably one double bond.

Preferably each R⁴ is an unsubstituted alkylene group.

Each R⁴ has at least 1 carbon atom. Suitably each R 4 has up to 18 carbon atoms, preferably up to 16 carbon atoms, more preferably up to 14 carbon atoms. In preferred embodiments each R⁴ has from 1 to 12 carbon atoms, preferably from 1 to 8 carbon atoms, for example from 1 to 4 carbon atoms. Each R⁴ may be straight chain or branched. R⁴ may be cyclic.

In some preferred embodiments each R⁴ is an unsubstituted alkylene group having 1 to 12, preferably 1 to 6, for example 2, 3 or 4 carbon atoms.

Each R⁴ may be the same or different.

In some embodiments n is 1, R⁵ is H and component (b) comprises a compound of formula (IA):

HX¹—R⁴—X²H (IA)

In some embodiments X¹ is 0 and X² is NH.

In some embodiments X¹ and X² are both O.

In some embodiments X¹ and X² are both NH.

In some embodiments each X is NH, n is more than 1, R⁵ is H and component (b) comprises a polyamine, suitably a polyalkylene polyamine.

Preferred polyalkylene polyamines are polyethylene polyamine comprising 1 to 12 ethylene groups.

In some embodiments each X is NH, n is 1, R⁵ is H and component (b) comprises a diamine, for example an alkylene diamine.

In some embodiments each X is O, n is 1, R⁵ is H and component (b) comprises a diol.

In some embodiments each X is O, n is 1, R⁵ is H, R⁴ is a hydroxy substituted alkylene group and component (b) comprises a polyol, for example a sugar or a sugar alcohol. Examples of suitable sugars include monosaccharides, disaccharides, and polysaccharides.

In some embodiments each X is O, R⁵ is H and component (b) comprises a glycol, suitably a polyalkylene glycol. Examples of suitable polyalkylene glycols include polyethylene glycol, polypropylene glycol, polybutylene glycol, and polytetramethylene ether glycol.

In some embodiments each terminal X is NH, all other groups X are O, R⁵ is H and component (b) comprises a polyetheramine.

Preferably in the compound of formula (I) each X is O, R⁵ is H and each R⁴ is CH₂CH₂, CH(CH₃)CH₂ (or CH₂CH(CH₃)), CH(CH₂CH₃)CH₂ (or CH₂CH(CH₂CH₃)) or CH₂CH₂CH₂CH₂.

Preferred polyalkylene glycols for use herein have a weight average molecular weight (Mw) of at least 100 g/mol, suitably at least 200 g/mol, for example at least 300 g/mol, for example at least 400 g/mol. Suitably the polyalkylene glycol has a weight average molecular weight of less than 3000 g/mol, suitably less than 2500 g/mol, for example less than 2200 g/mol. In preferred embodiments the polyalkylene glycol has a weight average molecular weight of from 100 to 3000 g/mol, preferably from 200 to 2500 g/mol, for example from 400 to 1500 g/mol.

In some embodiments the polyalkylene glycol may comprise a mixture of ethylene oxide derived units and propylene oxide derived units.

In some embodiments R⁵ is not hydrogen.

In such embodiments R⁵ is an optionally substituted alkyl, alkenyl, aryl, alkaryl or aralkyl group. Preferably R⁵ is an unsubstituted alkyl, alkenyl, aryl, alkaryl or aralkyl group, more preferably an unsubstituted alkyl or alkenyl group.

In some embodiments R⁵ is an unsubstituted alkyl group.

In some embodiments R⁵ is an unsubstituted alkenyl group. Suitably the alkenyl group may comprise one or more double bonds, for example one or two double bonds, suitably one double bond.

R⁵ suitably has at least 1 carbon atom. Suitably R⁵ has up to 22 carbon atoms, preferably up to 20 carbon atoms, more preferably up to 18 carbon atoms. In preferred embodiments R⁵ has from 1 to 18 carbon atoms, preferably from 1 to 12 carbon atoms, for example from 1 to 8 carbon atoms. R⁵ may be straight chain or branched.

In some embodiments n is 0 and component (b) comprises a compound of formula (IB):

R⁵—XH (IB)

wherein X and R⁵ are as defined above.

In some embodiments component (b) comprises an allylic alcohol, suitably allyl alcohol or an allyl alcohol alkoxylate. Examples of suitable allyl alcohol alkoxylates include allyl alcohol ethoxylate and allyl alcohol propoxylate.

In some embodiments component (b) comprises an alkoxylated compound, especially ethoxylated and/or propoxylated compounds.

Suitable alkoxylated compounds include polyalkylene glycols described above. Other suitable alkoxylated compounds include alkoxylated amines and diamines, alkoxylated alkanolamines and alkoxylated alcohols. Such compounds will be known to the person skilled in the art and are often available from a commercial source.

Component (b) may comprise a mixture of compounds.

In some embodiments component (b) may comprise a compound of formula (I) wherein R⁵ is hydrogen and a compound of formula (I) wherein R⁵ is not hydrogen, for example wherein R⁵ is an unsubstituted alkyl or alkenyl group.

In some embodiments component (b) is obtained at least partially from renewable sources. In some preferred embodiments component (b) is obtained entirely from renewable sources.

### Components (a) and (b)

In some preferred embodiments component (a) comprises an epoxidised fatty acid ester, and component (b) comprises a compound including at least two reactive alcohol and/or amino functional groups.

In some preferred embodiments component (a) comprises a methyl ester or 2-ethylhexyl ester of an epoxidised fatty acid and component (b) comprises a polyalkylene glycol compound including at least two reactive primary alcohol and/or primary amino functional groups.

In some preferred embodiments component (a) comprises an epoxidised soybean oil methyl ester and component (b) is selected from polyethylene glycol, polytetramethylene ether glycol, polypropylene glycol, polypropylene glycol diamine and allyl alcohol ethoxylate.

To form the surfactant of the present invention component (a) and component (b) are suitably reacted in a ratio of from 5:1 to 1:20, suitably from 3:1 to 1:10, preferably from 2:1 to 1:5, preferably from 1.5:1 to 1:3, for example from 1:1 to 1:2.

Component (a) and component (b) are suitably reacted under substantially anhydrous conditions. Water produced during the reaction may be removed. Performing such a reaction and selecting appropriate conditions will be within the competence of the person skilled in the art.

Suitably component (a) and component (b) are reacted in the presence of a catalyst. Any suitable catalyst may be used and the selection of a catalyst will be within the competence of the skilled person. Suitable catalysts include Lewis acid catalysts. Suitable catalysts include boron compounds, for example boron trifluoride or boron trichloride. One preferred catalyst is boron trifluoride etherate.

Suitably the reaction of component (a) and component (b) is carried out at a temperature below 200° C., preferably below 150° C., for example below 100° C. Suitably the reaction temperature is maintained between 80 and 20° C.

As the skilled person will appreciate, the reaction product of an epoxidised fatty acid ester (a) and amino/alcohol compound (b) will depend on the nature of components (a) and (b), the ratio of components used and the reaction conditions. In preferred embodiments in which component (b) comprises a compound including at least 2 amino or alcohol functional groups, a mixture of compounds may be formed.

The surfactant formed by the reaction of components (a) and (b) may include a compound of formula (IIA):

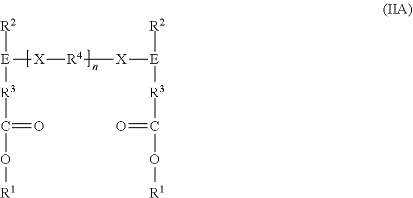

which is formed by the reaction of one molecule of component (b) and two molecules of component (a).

The surfactant formed by the reaction of components (a) and (b) may include a compound of formula (IIB):

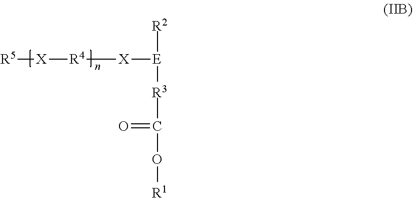

which is formed by the reaction of one molecule of component (a) and one molecule of component (b).

The surfactant formed by the reaction of components (a) and (b) may include a compound of formula (IIC):

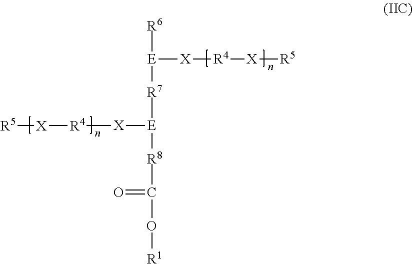

which is formed by the reaction of one molecule of component (a) and two molecules of component (b).

The skilled person will appreciate that in embodiments in which R⁵ is not hydrogen, compounds of formula (IIA) will not form.

In embodiments in which R⁵ is hydrogen, and the ester includes more than one epoxy functional group, more complex structures may also be formed, for example as shown in formulae (IID), (IIE) and (IIF):

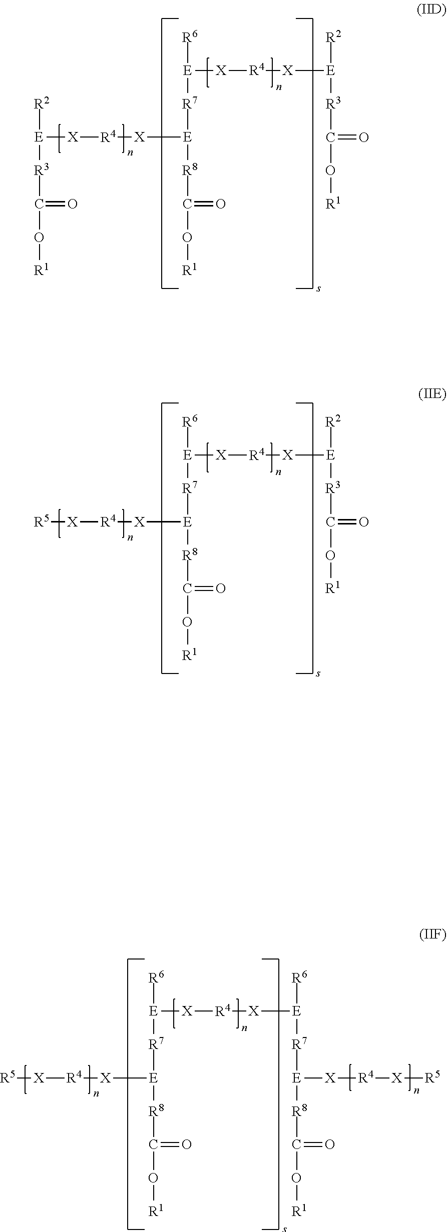

In each of structures (IIA), (IIB), (IIC), (IID), (IIE) and (IIF), E is the residue of an epoxy group having the structure

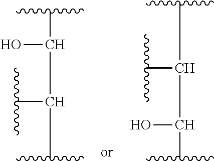 [SMILES: *C(*)C(*)O ; *C(*)C(*)O]

Suitably R^{2 ,} R^{3 ,} R^{6 ,} R^{7 ,} and R⁸ are each independently optionally substituted hydrocarbyl groups, preferably unsubstituted straight chain hydrocarbyl groups. In preferred embodiments R² and R³ together have a total of from 8 to 20 carbon atoms, and R^{6 ,} R^{7 ,} and R⁸ together have a total of from 6 to 18 carbon atoms.

s is preferably from 1 to 4.

X, R¹, R⁴, R⁵, and n are as previously defined herein.

The surfactant will typically comprise a mixture of compounds having the above structures depending on the starting compounds, the ratios reacted and the reaction conditions.

In some embodiments the present invention may provide a surfactant prepared from components (a) and (b) which is further reacted.

In some embodiments of the first or second aspects, the surfactant is the reaction product of:
(a) an epoxidised carboxylic acid ester;
(b) a compound including at least one reactive alcohol and/or amino functional group; and
(c) a derivatising agent.

Therefore the surfactant may be a derivatised surfactant. The derivatised surfactant may be prepared by:
(i) reacting (a) an epoxidised carboxylic acid ester with (b) a compound including at least one reactive alcohol and/or amino functional group; and
(ii) reacting the reaction product of step (i) with (c) a derivatising agent.

Components (a) and (b) are suitably as defined in relation to the first and second aspect.

Component (c) suitably comprises a compound which can react with a functional group present in the reaction product of step (i).

The derivatising agent (c) is a compound capable of reacting with a functional group present in the reaction product of component (a) and component (b).

Functional groups which may be present in the reaction product of component (a) and component (b) include esters, alkenes, hydroxy, epoxy and amino groups.

These functional groups may be present within the residue of component (a), for example an ester functional group or an alkene or residual epoxy group.

These functional groups may alternatively or additionally be present in the residue of component (b), for example a hydroxy or amino group. When component (b) comprises a compound of formula R⁵ -[X-R^{4 ]}-XH and R⁵ is not hydrogen, R⁵ may contain a functional group, for example a halide or alkene moiety. In some preferred embodiments R⁵ comprises a terminal alkene group.

The derivatising agent (c) is suitably selected depending on the functional group present in the reaction product of component (a) and component (b).

Suitably the derivatising agent includes a first functional group able to react with the reaction product of component (a) and component (b) and a second functional group.

In some embodiments the derivatising agent (c) may undergo an addition reaction with the reaction product of component (a) and component (b), for example to a double bond.

Suitably the reaction with component (c) introduces a polar functional group into the derivatised surfactant. This polar functional group may be selected from anionic, non-ionic or cationic functional groups.

The polar functional group may be selected from a sulfonate moiety, a sulfate moiety, a carboxylate moiety, a quaternary ammonium moiety, a phosphonate moiety, a phosphate moiety, a hydroxy group, an amino group and/or an alkoxylated chain.

In some embodiments component (c) comprises a hydrolysis agent. Such compounds may hydrolyse an ester moiety within the reaction product of component (a) and component (b), suitably the ester residue of component (a). The species formed suitably comprises a carboxylate moiety. Conditions for carrying out such reactions will be well known to the skilled person.

Suitable hydrolysis agents will be known to the skilled person. Hydrolysis may be carried out under basic or acidic conditions. Examples of suitable basic hydrolysis agents include sodium hydroxide, potassium hydroxide and ammonium hydroxide. Acidic hydrolysis may be carried out in the presence of any strong acid. Neutralisation may follow to provide an alkali metal, amine or ammonium salt.

In some embodiments component (c) comprises a compound including at least one reactive alcohol or amino functional group.

Such compounds may react with a substituted leaving group within the residue of the epoxidised carboxylic acid ester (a) or component (b). Suitably such groups undergo a trans-esterification or amidation reaction with the ester residue of component (a). Conditions for carrying out such reactions will be well known to the skilled person.

In embodiments where component (c) includes a compound having a reactive alcohol functional group, the reactive alcohol functional group is suitably a primary alcohol group or a secondary alcohol group. In preferred embodiments, the reactive alcohol functional group is a primary alcohol group.

In embodiments where component (c) includes a compound having a reactive amino functional group, the reactive amino functional group is suitably a primary amino group or a secondary amino group. In preferred embodiments, the reactive amino functional group is a primary amino group.

In some embodiments component (c) comprises a compound of formula (III):

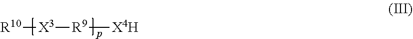

wherein p is 0 or a positive integer, each X³ is independently O or NR^{11 ,} X⁴ is O or NR^{12 ,} each R⁹ is independently an optionally substituted alkylene, alkenylene or arylene group, and each of R¹⁰, R¹¹ and R¹² is hydrogen or an optionally substituted alkyl, alkenyl, aryl, alkaryl or aralkyl group.

Preferably p is a positive integer.

In some embodiments p is no more than 300, suitably no more than 250, preferably no more than 200, preferably no more than 150, for example no more than 100.

In some embodiments p is 1.

In some embodiments p is 1 to 20.

In some embodiments p is 10 to 200, for example 10 to 100 or 10 to 50.

Each X³ may be the same or different.

In some preferred embodiments R¹¹ is H and X³ is O or NH.

In some embodiments, each X³ is O or NH. In some embodiments, at least one X³ is O and at least one X³ is NH. In preferred embodiments each X³ is O.

In some embodiments each X³ is O and X⁴ is O. In some embodiments each X³ is O and X⁴ is NH.

Each R⁹ is an optionally substituted alkylene, alkenylene or arylene group. Preferably each R⁹ is an unsubstituted alkylene, alkenylene or arylene group, more preferably an unsubstituted alkylene or alkenylene group.

When R⁹ is substituted, preferred substituents are amino and hydroxy substituents.

In some embodiments each R⁹ is an unsubstituted alkenylene group. Suitably the alkenylene group may comprise one or more double bonds, for example one or two double bonds, suitably one double bond.

Preferably each R⁹ is an unsubstituted alkylene group.

Each R⁹ has at least 1 carbon atom. Suitably each R⁹ has up to 18 carbon atoms, preferably up to 16 carbon atoms, more preferably up to 14 carbon atoms. In preferred embodiments each R⁹ has from 1 to 12 carbon atoms, preferably from 1 to 8 carbon atoms, for example from 1 to 4 carbon atoms. Each R⁹ may be straight chain or branched. R⁹ may be cyclic.

In some preferred embodiments each R⁹ is an unsubstituted alkylene group having 1 to 12, preferably 1 to 6, for example 2, 3, or 4 carbon atoms.

Each R⁹ may be the same or different.

R¹⁰ is hydrogen or an optionally substituted alkyl, alkenyl, aryl, alkaryl or aralkyl group. Preferably R¹⁰ is not hydrogen. Preferably R¹⁰ is an unsubstituted alkyl, alkenyl, aryl, alkaryl or aralkyl group, more preferably an unsubstituted alkyl or alkenyl group.

When R¹⁰ is substituted, hydroxy substituents are preferred.

In some embodiments R¹⁰ is an unsubstituted alkenyl group. Suitably the alkenyl group may comprise one or more double bonds, for example one or two double bonds, suitably one double bond.

In preferred embodiments R¹⁰ is an alkyl group. Preferred alkyl groups are unsubstituted alkyl groups and hydroxy substituted alkyl groups. Unsubstituted alkyl groups are especially preferred.

R¹⁰ has at least 1 carbon atom. Suitably R¹⁰ has up to 22 carbon atoms, preferably up to 20 carbon atoms, more preferably up to 18 carbon atoms. In preferred embodiments R¹⁰ has from 1 to 18 carbon atoms, preferably from 1 to 12 carbon atoms, for example from 1 to 8 carbon atoms. R¹⁰ may be straight chain or branched.

In some embodiments component (c) comprises an alkoxylated compound.

Suitable alkoxylated compounds include alkoxy polyalkylene glycols and alkoxy polyalkylene glycol amines, wherein the polyalkylene glycol portion is preferably selected from polyethylene glycol, polypropylene glycol, polybutylene glycol, polytetramethylene ether glycol, and copolymers thereof, and the alkoxy portion is preferably selected from methoxy, ethoxy, propoxy, and butoxy.

Examples of suitable alkoxy polyalkylene glycols and alkoxy polyalkylene glycol amines include methoxy polyethylene glycol, butoxy polypropylene glycol, and butoxy poly(ethylene glycol-co-propylene glycol) amine.

In some embodiments p is 1, R¹⁰ is hydrogen, X³ is O or NH and X⁴ is O, NH or NR¹² wherein R¹² is preferably an alkyl group or a hydroxy alkyl group.

In some embodiments R¹⁰ is alkyl, preferably unsubstituted alkyl or hydroxy alkyl, p is 0 and X⁴ is NR¹² wherein R¹² is alkyl, preferably unsubstituted alkyl or hydroxy alkyl.

In some embodiments component (c) may be a primary or secondary amine, for example an alkyl amine, a dialkyl amine, an alkanolamine or a dialkanolamine. Such compounds typically include 1 to 12 carbon atoms in each alkyl or hydroxy alkyl group, preferably from 1 to 8 carbon atoms, most preferably from 1 to 4 carbon atoms.

Examples of suitable compounds of this type include monoethanolamine, monoisopropanolamine, diethanolamine, and diisopropanolamine.

In some embodiments R¹⁰ is alkyl, preferably C₁ to C₁₄ alkyl, X³ is NR¹¹ and R¹¹ is alkyl, preferably C₁ to C₁₄ alkyl, R⁹ is alkylene and X⁴ is NH or O.

Such compounds may react to form a species which further includes a tertiary amine. This tertiary amine group can be reacted with a quaternising agent to introduce cationic functionality into the molecule.

Examples of suitable compounds of this type include dimethylaminopropylamine (DMAPA), dimethylaminopropanol and dimethylaminoethanol.

Other suitable compounds of this type will be known to the person skilled in the art.

In some embodiments, the derivatised surfactant is prepared by:
(i) reacting (a) an epoxidised carboxylic acid ester with (b) a compound including at least one reactive alcohol and/or amino functional group;
(ii) reacting the reaction product of step (i) with (c) a derivatising agent comprising a compound including at least one reactive alcohol or amino functional group and one tertiary amino group; and
(iii) reacting the reaction product of step (ii) with (d) a quaternising agent.

Suitable quaternising agents will be known to the skilled person. Examples of suitable quaternising agents include dimethyl sulfate, diethyl sulfate, methyl chloride, methyl bromide, benzyl chloride, monochloroacetic acid and epoxides in combination with an acid.

In embodiments where the reaction product of component (a) and component (b) comprises an alkene functional group component (c) may comprise a sulfonating agent.

Suitable sulfonating agents will be known to the skilled person. Examples of suitable sulfonating agents include sodium bisulfite, sodium metabisulfite, potassium bisulfite, and potassium metabisulfite.

Selection of suitable reaction conditions and ratios of reagents for carrying out step (ii) of the method will be within the competence of the skilled person.

In some embodiments a catalyst will be needed.

Suitable catalysts will be known to the person skilled in the art.

The mild surfactant composition The mild surfactant composition of the first and second aspects of the present invention suitably comprises one or more further components.

The amount of surfactant present in the composition will depend on the intended use of the composition and the presence of other components.

In some embodiments, the mild surfactant composition comprises a surfactant, which is a reaction product as described above, and a diluent or carrier.

Preferably the mild surfactant composition is an aqueous composition. Preferably water is the main solvent present in the composition. By this we mean that water preferably accounts for at least 50 w t% of all solvents present in the composition, preferably at least 60 wt %, more preferably at least 70 wt %, for example at least 80 wt % or at least 90 wt %.

The mild surfactant composition may further comprise one or more additional solvents. Preferred solvents are water miscible solvents, for example alcohols.

In some embodiments, the mild surfactant composition further comprises a pigment.

In some embodiments, the mild surfactant composition is a detergent composition.

In some embodiments, the mild surfactant composition is a laundry composition.

In some embodiments, the mild surfactant composition is a hand dishwashing composition.

In some embodiments, the mild surfactant composition is a personal care composition, for example a shampoo, a hair conditioner, a body wash, a hand wash or a cleanser.

In some embodiments, the mild surfactant composition is a toiletries composition.

In some embodiments, the mild surfactant composition is a cosmetic composition.

In some embodiments, the mild surfactant composition is an animal care composition.

Suitably the mild surfactant composition comprises the reaction products described above as the main surfactant in said composition. In some embodiments, the mild surfactant composition may contain other mild surfactants.

The surfactants used in the present invention may replace a non-mild surfactant in a known surfactant composition, such as a laundry composition or a personal care composition, to improve the mildness of the surfactant composition and suitably form a mild surfactant composition according to the first aspect. The surfactants used in the present invention may replace a mild surfactant in a known mild surfactant composition, such as a laundry composition or a personal care composition, to provide an alternative or improved mild surfactant composition of the first aspect, suitably having an improved environmental profile due to the surfactants of the present invention being obtained from renewable sources.

A person skilled in the relevant art would be able to carry out any reformulation of such known surfactant compositions or known mild surfactant compositions which would be required to include the surfactants of the present invention.

The invention will now be further described with reference to the following non-limiting examples.

### EXAMPLES

### Example 1—Reaction of Epoxidised Methyl Soyate (Soybean Oil Methyl Ester) with Polyethylene Glycol 200 Molecular Weight

284.8 grams of epoxidised methyl soyate with an epoxide value of 227 mg KOH/g was placed into a flask. 115.2 grams of 200 molecular weight polyethylene glycol (1:1 equivalents epoxy to hydroxy) was then added. The mixture was dried to a water content of <0.1%, then cooled to 20° C. 0.32 grams of boron trifluoride etherate catalyst was added, and the reaction was cooled to maintain the temperature below 60° C. The reaction was continued until the epoxide value of the mixture was <1 mg KOH/g.

### Example 2—Reaction of Epoxidised Methyl Soyate with Polyethylene Glycol 200 Molecular Weight

221.1 grams of epoxidised methyl soyate with an epoxide value of 227 mg KOH/g was placed into a flask. 178.9 grams of 200 molecular weight polyethylene glycol (1:2 equivalents epoxy to hydroxy) was then added. The mixture was dried to a water content of <0.1%, then cooled to 20° C. 0.32 grams of boron tetrafluoride etherate catalyst was added, and the reaction was cooled to maintain the temperature below 60° C. The reaction was continued until the epoxide value of the mixture was <1 mg KOH/g.

### Example 3—Reaction of Epoxidised Methyl Soyate with Polyethylene Glycol 600 Molecular Weight

180.7 grams of epoxidised methyl soyate with an epoxide value of 227 mg KOH/g was placed into a flask. 219.3 grams of 600 molecular weight polyethylene glycol (1:1 equivalents epoxy to hydroxy) was then added. The mixture was dried to a water content of <0.1%, then cooled to 30° C. 0.32 grams of boron tetrafluoride etherate catalyst was added, and the reaction was cooled to maintain the temperature below 60° C. The reaction was continued until the epoxide value of the mixture was <1 mg KOH/g.

### Example 4—Reaction of Epoxidised Methyl Soyate with Polyethylene Glycol 1500 Molecular Weight

99.1 grams of epoxidised methyl soyate with an epoxide value of 210 mg KOH/g was placed into a flask. 300.9 grams of 1500 molecular weight polyethylene glycol (1:1 equivalents epoxy to hydroxy) was then added. The mixture was dried to a water content of <0.1%, then cooled to 60° C. 0.32 grams of boron tetrafluoride etherate catalyst was added, and the reaction was cooled to maintain the temperature below 80 ° C. The reaction was continued until the epoxide value of the mixture was <1 mg KOH/g.

Example 5 - Reaction of epoxidised methyl soyate with polytetrahydrofuran 650 molecular weight 172.8 grams of epoxidised methyl soyate with an epoxide value of 210 mg KOH/g was placed into a flask. 227.2 grams of 650 molecular weight polytetrahydrofuran (1:1 equivalents epoxy to 5 hydroxy) was then added. The mixture was dried to a water content of <0.1%, then cooled to 60° C. 0.32 grams of boron tetrafluoride etherate catalyst was added, and the reaction was cooled to maintain the temperature below 80° C. The reaction was continued until the epoxide value of the mixture was <1 mg KOH/g.

Example 6 - Reaction of epoxidised methyl soyate with a 400 molecular weight polyether 10 diamine based on polypropylene glycol 213.8 grams of epoxidised methyl soyate with an epoxide value of 210 mg KOH g was placed into a flask. 186.2 grams of 400 molecular weight polyether diamine (1:1 equivalents epoxy to amine) was then added. With a water content of <0.1% the reaction mixture was heated to 120 to 140° C., and the reaction was continued until the epoxide value of the mixture was <1 mg 15 KOH/g.

Example 7 - Reaction of epoxidised methyl soyate with 500 molecular weight allyl alcohol ethoxylate 132.3 grams of epoxidised methyl soyate with an epoxide value of 210 mg KOH/g was placed into a flask. 267.9 grams of 500 molecular weight allyl alcohol ethoxylate (1:1 equivalents epoxy 20 to hydroxy) was added. The mixture was dried to a water content of <0.1%, then cooled to 60° C. 0.32 grams of boron tetrafluoride etherate catalyst was added, and the reaction was cooled to maintain the temperature below 80° C. The reaction was continued until the epoxide value of the mixture was <1 mg KOH/g.

Example 8 - Derivatisation of Example 3 by transesterification of the ester group with a methyl 25 initiated poly ethylene glycol of 500 molecular weight (MPEG500) 231.5 grams of Example 3 was placed in a flask. 168.5 grams of MPEG500 was then added. The mixture was dried to a water content of <0.1%, then cooled to 60° C. 1% of 20% sodium methoxide catalyst was then added, and vacuum was applied to distil the methanol from the catalyst and by-product from the reaction. The reaction mixture was slowly heated to a maximum 30 of 90° C. until distillation stopped.

Example 9 - Derivatisation of Example 3 by transesterification of the ester group with a methyl initiated poly ethylene glycol of 1000 molecular weight (MPEG1000) 162.9 grams of Example 3 was placed in a flask. 237.1 grams of MPEG1000 was then added. The mixture was dried to a water content of <0.1%, then cooled to 60° C. 1% of 20% sodium methoxide catalyst was then added, and vacuum was applied to distil the methanol from the catalyst and by-product from the reaction. The reaction mixture was slowly heated until distillation stopped.

### Example 10—Derivatisation of Example 3 by Amidation of the Ester Group with a Butyl Initiated Polyethylene/Polypropylene Glycol of 2000 Molecular Weight Polyether Amine

102.2 grams of Example 3 was placed in a flask. 297.8 grams of polyether amine was then added. The mixture was dried to a water content of <0.1%, then cooled to 60° C. 1% of 20% sodium methoxide catalyst was then added, and vacuum was applied to distil the methanol from the catalyst and by-product from the reaction. The reaction mixture was slowly heated to a maximum of 90° C. until distillation stopped.

### Example 11—Derivatisation of Example 5 by Transesterification of the Ester Group with a Butyl Initiated Polypropylene Glycol of 1200 Molecular Weight (BPPG1200)

150.3 grams of Example 5 was placed in a flask. 249.7 grams of BPPG1200 was then added. The mixture was dried to a water content of <0.1%, then cooled to 60° C. 1% of 20% sodium methoxide catalyst was then added, and vacuum was applied to distil the methanol from the catalyst and by-product from the reaction. The reaction mixture was slowly heated to a maximum of 90° C. until distillation stopped.

### Example 12—Derivatisation of Example 3 by Amidation of the Ester Group with Diethanolamine

102.2 grams of Example 3 was placed in a flask. 297.8 grams of diethanolamine was then added. The mixture was dried to a water content of <0.1%, then cooled to 60° C. 1% of 20% sodium methoxide catalyst was then added, and vacuum was applied to distil the methanol from the catalyst and by-product from the reaction. The reaction mixture was slowly heated to a maximum of 90° C. until distillation stopped. The reaction mixture was then allowed to equilibrate at 60° C. for 3 hours.

Example 13—Derivatisation of Example 7 by Sulfonation Using Sodium Bisulfate

89.9 grams of Example 7 was placed in a flask with 279.5 grams of water. 29.5 grams of sodium metabisulfite was then added with 1.1 grams of a 50% sodium hydroxide solution, and the reaction mixture was slowly heated to 90-95° C. The reaction was monitored by anionic surfactant content and was continued until the value became constant.

### Example 14—Derivatisation of Example 1 by Amidation of the Ester Group with Dimethylaminopropylamine (DMAPA)

312.4 grams of Example 1 was placed in a flask and dried to <0.1% water content. 87.6 grams of DMAPA was then added (10% molar excess). The temperature was adjusted to 60° C., then 1% of 20% sodium methoxide catalyst was added, and the reaction mixture was slowly heated to 140° C. to distil the methanol from the catalyst and by-product from the reaction until distillation stopped. Once methanol distillation stopped, vacuum was applied and the reaction mixture was heated to 150° C. until the distillation of the excess DMAPA was complete.

### Example 15—Derivatization of Example 14 to Form a Betaine by Reaction with Monochloroacetic Acid

110 grams of Example 14 was charged to a flask along with 264.9 grams of water. 25.1 grams of monochloroacetic acid was then added. The pH was adjusted to 9.5-10.5 with 50% sodium hydroxide solution, and the reaction mixture was heated to 85° C. With the pH being maintained in the range 9.5-10.5, the reaction was continued until the base value was constant and <5.0 mg KOH/g.

### Example 16—Reaction of Epoxidised Methyl Soyate with Polyethylene Glycol 200 Molecular Weight

221.1 grams of epoxidised soyate with an epoxide value of 227 mg KOH / g was placed into a flask. 178.9 grams of 200 molecular weight polyethylene glycol (1:2 equivalents epoxy to hydroxy) was then added. The mixture was dried to a water content of <0.1%, then cooled to 20° C. 0.32 grams of boron tetrafluoride etherate catalyst was added, and the reaction was cooled to maintain the temperature below 60° C. The reaction was continued until the epoxide value of the mixture was <1 mg KOH/g. The final product had a saponification value of 129.2 mg KOH/g. 110 grams of this material was dispersed in 277.8 grams of water, and 12.2 grams of sodium hydroxide (a 20% molar excess of the quantity required for full hydrolysis) was then added. This mixture was heated to 95° C. for 12 hours, after which the reaction mixture was neutralized to pH 10.5 with acetic acid.

### Example 17—Derivatization of Example 4 by Transesterification of the Ester Group with a Methyl Initiated Poly Ethylene Glycol of 1000 Molecular Weight (MPEG1000)

192.0 grams of Example 4 was placed in a flask, 208 grams of MPEG1000 was then added, the mixture was dried to a water content of <0.1%, before cooling to 60° C. 1% of 20% sodium methoxide catalyst was then added, vacuum was applied to distil the methanol from the catalyst and by product from the reaction, the reaction mixture was slowly heated until distillation stopped.

### Example 18—Derivatization of Example 3 by Amidation of the Ester Group with Dimethylaminopropylamine (DMAPA)

342.8 grams of Example 3 was placed in a flask and dried to <0.1% water content, 57.2 grams of DMAPA was then added (10% molar excess), the temperature was adjusted to 60° C. before the addition of 1% of 20% sodium methoxide catalyst, the reaction mixture was slowly heated to 140° C. to distil the methanol from the catalyst and by product from the reaction until distillation stopped. Once methanol distillation stopped vacuum was applied and the reaction mixture was heated to 150° C. until the distillation of the excess DMAPA was complete.

### Experimental—Skin Irritation Testing

Skin irritation is defined as reversible damage to the skin following exposure to a single substance or a composition for up to 4 hours.

The irritancy of the surfactants described herein was evaluated using a Reconstructed Human Epidermis ET50 method which is an adaptation of a regulatory skin irritation test. Subtle differences between mild and ultra-mild products and formulations can be measured using this test, allowing a series of products to be placed into rank order of irritation potential and therefore mildness. This method enables the irritation potential of the surfactant under test to be classified as “severe”, “moderate”, “mild” or “minimal/non-irritating” and can be useful in classifying a series of products or ingredients in rank order of skin irritation potential. The rank order is assigned based on the ET50 values, which can also be benchmarked against competitor products or historical data for specific product types.

The test method is based on reconstructed human epidermis (RhE) which mimics the biochemical and physiological properties of the upper layers of human skin. The test substance is applied directly to the RhE surface, providing a good model of “real life” exposure. Cell viability is measured by enzymatic conversion of the vital dye MTT into a blue formazan salt that is quantitatively measured after extraction from the RhE tissues. Irritation potential is calculated in terms of the “ET50” value: the time taken, in minutes, for the test item to reduce the viability of the skin model to 50% compared with negative controls. ET50 values are then used to assign the irritancy classification based on a proven prediction model and, where relevant, to place a series of test substances into a rank order of irritation potential.

The relationship of ET50 value to irritancy classification is shown in Table 1 below.

TABLE 1

| ET50 (h) | Expected In vivo Irritancy |
|---|---|
| <0.5 | Strong/severe possible corrosive concentration |
| 0.5-4 | Moderate |
| 4-12 | Moderate to mild |
| 12-24 | Very mild |
| >24 | Non-irritating |

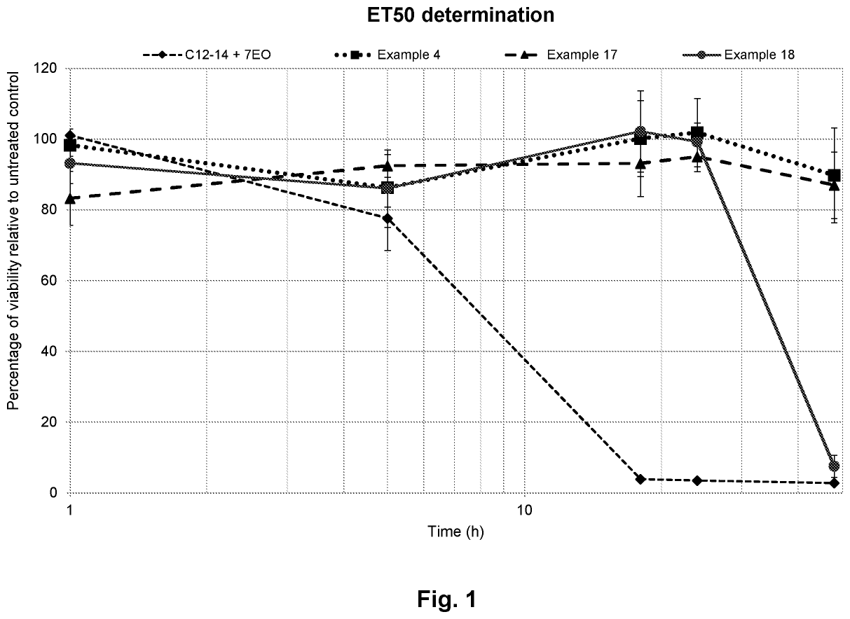

The surfactants to be evaluated were diluted to a concentration of 0.3 wt in aqueous solutions and the pH of these solutions was adjusted to pH 4.7. A typical non-ionic surfactant prepared from a C12-C14 alcohol ethoxylate and 7 equivalents of ethylene oxide was included as a comparative example. Such non-ionic surfactants are used in many household and industrial cleaning applications. The ET50 results for Examples 4, 17 and 18, as well as the comparative example, are shown in FIG. 1 and are summarised below in Table 2.

TABLE 2

| Sample | ET50 (Hours) | Classification |
|---|---|---|
| C12-14 + 7 EO | 8.08 | Moderate to mild |
| Example 4 | >48 | Non-irritating |
| Example 17 | >48 | Non-irritating |
| Example 18 | 34.8 | Non-irritating |

These results show Examples 4, 17 and 18 are classified as “non-irritating” and therefore extremely mild. The conventional C12-14+7EO surfactant which is commonly used in a wide range of applications was found to be more irritating, classified as “moderate to mild” irritation. Both Examples 4 and 17 showed no onset of irritancy within the 48 hours' time period of the test.

Another interesting observation is the classification of Example 18 as “non-irritating”. Typically, a relatively low molecular weight non-ionic surfactant containing tertiary amine groups tend to be irritating. In this case the functionality and structure of the molecule appears to help to reduce the expected irritancy levels.

For reference, typical ET50 irritancy results for anionic and amphoteric surfactants commonly used in personal care and household cleaning products are shown in Table 3 below.

TABLE 3

| Sample | ET50 (Hours) | Classification |
|---|---|---|
| Sodium laureth sulfate | 10.25 | Moderate to mild |
| Sodium lauryl sulfate | 9.37 | Moderate to mild |
| Cocamidopropyl betaine | 29.4 | Non-irritating |

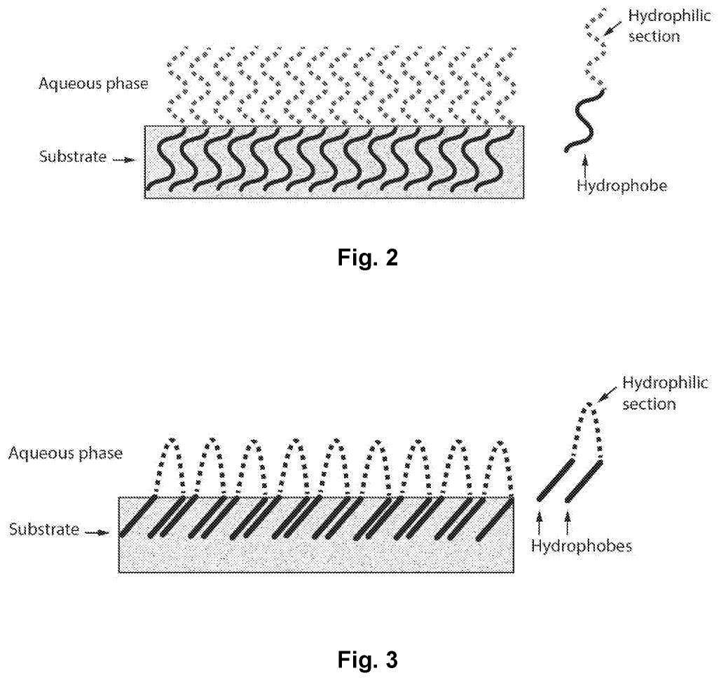

Without being bound by theory, we believe that an important factor which determines the degree of irritancy and level of mildness of a surfactant may be how the surfactant molecule packs onto a particular substrate, such as the skin or eye. A conventional non-ionic surfactant as depicted in FIG. 2 packs very tightly on the substrate with the hydrophobic sections attaching themselves to the substrate and the hydrophilic tails locating themselves in the aqueous phase. A typical example of a conventional non-ionic surfactant is the comparative example C12-14+7EO surfactant discussed above (see Table 2).

The mild surfactants of the present invention (such as Examples 4, 17 and 18) have a more structured form and pack onto a substrate in a more ordered and spaced-apart manner as depicted in FIG. 3. This packing helps to reduce the concentration of surfactant molecules across a given surface area and as the concentration of surfactant molecules is directly related to the degree of mildness, this type of packing can help reduce irritancy levels. Another important factor relating to the structure of the mild surfactants of the present invention is that the hydrophilic loop is located away from the substrate in a fixed loop, unlike in the conventional non-ionic surfactants discussed above. This means that the chances of the potentially irritating hydrophilic part coming into contact with the substrate (i.e. skin or an eye) is greatly reduced which therefore may significantly reduce the irritancy of the surfactants of the present invention.

In summary, the present invention provides a mild surfactant composition for use in personal care and/or detergent compositions. The composition comprises a surfactant which is mild and/or non-irritating to skin. The mild surfactant is the reaction product of (a) an epoxidised carboxylic acid ester; and (b) a compound including at least one reactive alcohol and/or amino functional group. The components (a) and (b) are suitably obtained from renewable sources. A use of such surfactants in mild surfactant compositions is also provided.

Although a few preferred embodiments have been shown and described, it will be appreciated by those skilled in the art that various changes and modifications might be made without departing from the scope of the invention, as defined in the appended claims.

Throughout this specification, the term “comprising” or “comprises” means including the component(s) specified but not to the exclusion of the presence of other components. The term “consisting essentially of” or “consists essentially of” means including the components specified but excluding other components except for materials present as impurities, unavoidable materials present as a result of processes used to provide the components, and components added for a purpose other than achieving the technical effect of the invention. Typically, when referring to compositions, a composition consisting essentially of a set of components will comprise less than 5% by weight, typically less than 3% by weight, more typically less than 1% by weight of non-specified components.

The term “consisting of” or “consists of” means including the components specified but excluding addition of other components.

Whenever appropriate, depending upon the context, the use of the term “comprises” or “comprising” may also be taken to encompass or include the meaning “consists essentially of” or “consisting essentially of”, and may also be taken to include the meaning “consists of” or “consisting of”.

For the avoidance of doubt, wherein amounts of components in a composition are described in wt %, this means the weight percentage of the specified component in relation to the whole composition referred to.

The optional features set out herein may be used either individually or in combination with each other where appropriate and particularly in the combinations as set out in the accompanying claims. The optional features for each aspect or exemplary embodiment of the invention as set out herein are also to be read as applicable to any other aspect or exemplary embodiments of the invention, where appropriate. In other words, the skilled person reading this specification should consider the optional features for each exemplary embodiment of the invention as interchangeable and combinable between different exemplary embodiments.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, and drawings), and/or all of the steps of any method or process so disclosed, may be combined in any combination, except combinations where at least some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, and drawings), or to any novel one, or any novel combination, of the steps of any method or process so disclosed.

1. A mild surfactant composition comprising a surfactant which is the reaction product of:
(a) an epoxidised carboxylic acid ester; and
(b) a compound including at least one reactive alcohol and/or amino functional group.

2. Use of a reaction product of:
(a) an epoxidised carboxylic acid ester; and
(b) a compound including at least one reactive alcohol and/or amino functional group, as a surfactant in a mild surfactant composition.

3. The composition or use according to claim 1, wherein component (a) comprises an ester of formula RCOOR¹ in which R is a hydrocarbyl group including an epoxy functional group and R¹ is a hydrocarbyl group.

4. The composition according to claim 3, wherein R is an unbranched aliphatic group having 6 to 26 carbon atoms and R¹ is methyl or 2-ethylhexyl.

5. The composition according to claim 1, wherein component (a) is derived from soybean oil fatty acid.

6. The composition or use according to claim 1, wherein component (b) comprises a compound of formula (I): 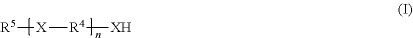
wherein n is 0 or a positive integer; each X is independently O or NH; each group R⁴ is independently a substituted alkylene, alkenylene or arylene group; and R⁵ is hydrogen or a substituted alkyl, alkenyl, aryl, alkaryl or aralkyl group provided that n is not 0 when R⁵ is hydrogen.

7. The composition according to claim 6, wherein component (b) comprises an alkoxylated compound.

8. The composition according to claim 7 wherein each Xis O, n is 20 to 100, R⁵ is hydrogen and each R⁴is a C2 to C4 alkylene group.

9. The composition according to claim 1, wherein the surfactant is the reaction product of:
(a) the epoxidised carboxylic acid ester;
(b) the compound including at least one reactive alcohol and/or amino functional group;
(c) a derivatising agent.

10. The composition or use according to claim 9 wherein reaction with component (c) introduces a polar functional group into the surfactant.

11. The composition according to claim 10 wherein the polar functional group is selected from anionic, non-ionic or cationic functional groups.

12. The composition according to claim 9 wherein component (c) comprises a compound of formula (III): 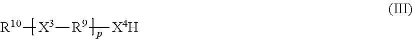
wherein p is 0 or a positive integer, each X³ is independently O or NR¹¹, X⁴ is O or NR¹², each R⁹ is independently an optionallya substituted alkylene, alkenylene or arylene group, and each of R¹⁰, R¹¹ and R¹² is hydrogen or a substituted alkyl, alkenyl, aryl, alkaryl or aralkyl group.

13. The composition according to claim 9 wherein component (c) comprises a sulfonating agent.

14. The composition according to claim 9 wherein component (c) comprises a hydrolysis agent.

15. The composition according to claim 1, wherein the mild surfactant composition is a mild personal care composition.

16. The composition or use according to claim 1, wherein the mild surfactant composition is a mild detergent composition.

17. The use according to claim 2, wherein component (a) comprises an ester of formula RCOOR¹ in which R is a hydrocarbyl group including an epoxy functional group and R¹ is a hydrocarbyl group.

18. The composition according to claim 2, wherein component (b) comprises a compound of formula (I): 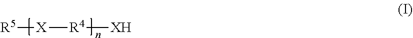
wherein n is 0 or a positive integer; each X is independently O or NH; each group R⁴ is independently a substituted alkylene, alkenylene or arylene group; and R⁵ is hydrogen or a substituted alkyl, alkenyl, aryl, alkaryl or aralkyl group provided that n is not 0 when R⁵ is hydrogen.

19. The composition according to claim 2, wherein the surfactant is the reaction product of:
(a) the epoxidised carboxylic acid ester;
(b) the compound including at least one reactive alcohol and/or amino functional group; and
(c) a derivatising agent.

20. The composition according to claim 19 wherein component (c) comprises a compound of formula (III): 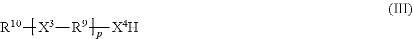
wherein p is 0 or a positive integer, each X³ is independently O or NR¹¹, X⁴ is O or NR¹², each R⁹ is independently a substituted alkylene, alkenylene or arylene group, and each of R^{1o}, R¹¹ and R¹² is hydrogen or a substituted alkyl, alkenyl, aryl, alkaryl or aralkyl group.
